(19) United States
(12) Patent Application Publication
(10) Pub. No.: US 2008/0085293 A1
(43) Pub. Date: Apr. 10, 2008

(54) Drug eluting stent and therapeutic methods using c-Jun N-terminal kinase inhibitor

(76) Inventor: Jenchen Yang, Diamond Bar, CA (US)

Correspondence Address: ROGER A. GILCREST, 250 WEST STREET, COLUMBUS, OH, 43216-7513, US

(21) Appl. No.: 11/507,743

(22) Filed: Aug. 22, 2006

(51) Int. Cl. A61F 2/02

(52) U.S. Cl. 424/423

(57) ABSTRACT

The present invention relates to a system and device for preventing stenosis and/or restenosis after an invasive procedure in a body vessel or cavity having an inner wall surface, the system comprising inserting a device coated with a growth arresting, lipid-derived, bioactive substance at a desired location along the inner wall surface of the body vessel or cavity. The present invention provides for the use of c-Jun aminoterminal kinase inhibitor (“JNK Inhibitor”) and certain analogs as restenosis inhibitors, incorporated into a stent.

### BACKGROUND OF THE INVENTION

Percutaneous coronary intervention (PCI) is used to treat obstructive coronary artery disease by compressing atheromatous plaque to the sides of the vessel wall. PCI is widely used with an initial success rate of over 90%. Approximately 1.2 million angioplasties were conducted in the United States alone in 2000. Despite the frequent application of this procedure and its high initial success rate, the long-term success of PCI is limited by intraluminal renarrowing or restenosis at the site of the procedure.

The American Heart Association in their 2006 Heart Disease and Stroke Statistics also show long term upward trends in the several main types of cardiovascular procedures and operations.

Vascular restenosis is a major long-term complication following surgical intervention of blocked arteries by percutaneous transluminal coronary angioplasty (PTCA), atherectomy, laser angioplasty and arterial bypass graft surgery. In about 35% of the patients who undergo PTCA, reocclusion occurs within three to six months after the procedure. The current strategies for treating vascular restenosis include mechanical intervention by devices such as stents or pharmacologic therapies including heparin, low molecular weight heparin, coumarin, aspirin, fish oil, calcium antagonist, steroids, and prostacyclin.

In-stent restenosis is believed to be due to neointimal hyperplasia (Serruys et al., 1994, N. Engl. J. Med., 331:489). Stent-induced mechanical arterial injury and a foreign-body response to the prosthesis are believed to result in acute and chronic inflammation in the vessel wall, leading to production of cytokines and growth factors (Serruys et al., 1994, N. Engl. J. Med., 331:489). These are believed to activate multiple signaling pathways, inducing vascular smooth muscle cell (VSMC) proliferation, which is believed to result in neointimal hyperplasia (Serruys et al., 1994, N. Engl. J. Med., 331:489). In addition to VSMC proliferation, VSMC migration and phenotypic differentiation, as well as extracellular matrix formation and degradation are believed to determine the extent of neointimal formation (Newby and George, 1996, Curr. Opin. Cardiol., 11:547). The predominant feature of late restenosis lesions is a large amount of extracellular matrix with a reduced number of smooth muscle cells, whereas in the early stages of intimal thickening formation the number of smooth muscle cells is increased (Pauletto et al., 1994, Clin. Sci., 87:467). To successfully prevent neointimal formation and restenosis, compounds that exert multifactorial effects on cellular activation and extracellular matrix constituents are likely to be necessary, and restenosis prevention using an approach that targets only one causative factor is believed to lack promise (Rosanio et al., 1999, Thromb. Haemost., 82(S1):164).

In the pathogenesis of restenosis excessive cell proliferation and migration occurs as a result of growth factors produced by cellular constituents in the blood and the damaged arterial vessel wall that mediate the proliferation of smooth muscle cells in vascular restenosis. Agents that inhibit the proliferation and/or migration of smooth muscle are useful in the treatment and prevention of restenosis. Further, agents that inhibit the inflammatory response of smooth muscle are useful in the treatment and prevention of restenosis.

Stent placement has largely supplanted balloon angioplasty because it is able to more widely restore intraluminal dimensions, which has the effect of reducing restenosis by approximately 50%. Ironically, stent placement actually increases neointimal growth at the treatment site, but because a larger lumen can be achieved with stent placement, the tissue growth is more readily accommodate, and sufficient luminal dimensions are maintained, so that the restenosis rate is nearly halved by stent placement compared with balloon angioplasty alone.

The pathophysiological mechanisms involved in restenosis are not fully understood. While a number of clinical, anatomical and technical factors have been linked to the development of restenosis, at least 50% of the process has yet to be explained. However, it is known that following endothelial injury, a series of repair mechanisms are initiated. Within minutes of the injury, a layer of platelets and fibrin is deposited over the damaged endothelium. Within hours to days, inflammatory cells begin to infiltrate the injured area. Within 24 hours after an injury, vascular smooth muscle cells (SMCs) located in the vessel media commence DNA synthesis. A few days later, these activated, synthetic SMCs migrate through the internal elastic lamina towards the luminal surface. A neointima is formed by these cells by their continued replication and their production of extracellular matrix. An increase in the intimal thickness occurs with ongoing cellular proliferation matrix deposition. When these processes of vascular healing progress excessively, the pathological condition is termed intimal hyperplasia or neointimial hyperplasia. Histological studies in animal models have identified neointimal hyperplasia as the central element in restenosis.

The responses to vascular injury that lead to restenosis have certain features in common with the processes leading to the development of the vascular lesions of atherosclerosis. Currently, it is understood that the lesions of atherosclerosis are initiated by some form of injury to arterial endothelium, whether due to hemodynamic factors, endothelial dysfunction or a combination of these or other factors (Schoen, “Blood vessels,” pp. 467-516 in Pathological Basis of Disease (Philadelphia: Saunders, 1994)). Inflammation has been implicated in the formation and progression of atherosclerotic lesions. Several inflammatory products, including IL-1.beta., have been identified in atherosclerotic lesions or in the endothelium of diseased coronary arteries (Galea, et al. (1996) Arterioscler Thromb Vasc Biol. 16:1000-6). Also, serum concentrations of IL-1.beta. are elevated in patients with coronary disease (Hasdai, et al. (1996) Heart, 76:24-8). Realizing the importance of inflammatory processes in the final common pathways of vascular response to injury allows analogies to be drawn between the lesions seen in restenosis and those seen in atherosclerosis.

Historically, approximately 1.2 million patients per year undergo PCI procedures. Restenosis and progressive atherosclerosis are the most common mechanisms for late failure in these reconstructions. Accordingly, there remains a need for devices and therapeutic methods to reduce restenosis as brought about by cell growth and inflammation that lead to arteriosclerosis.

Since the first performance of percutaneous transluminal coronary angioplasty (PTCA) in 1977, this procedure has become a widely accepted treatment modality for coronary artery disease (CAD) managing both single and multivessel disease.

However, all percutaneous techniques, regardless of the mode of intervention, have rather high rates of repeat interventions at long-term follow-up, representing a principle limitation of such a strategy. Stents appeared to the only device impacting, significantly, both acute and longterm outcome. Nevertheless, stents did not resolve the problem of restenosis which still occurs in at least 20-30% of the patients undergoing stent assisted percutaneous coronary interventions (PCI).

The advent of drug-eluting stents (DES) has dramatically reduced restenosis. A pooled analysis documented a 74% reduction in the risk of target lesion revascularization from the use of sirolimus-eluting stents (SES) (Cypher, Johnson & Johnson, Miami Lakes, Fla.) or paclitaxel-eluting stents (PES) (TAXUS, Boston Scientific Corp., Natick, Mass.) compared to bare-metal stents (BMS). However, certain subset of patient still has significant restenosis rate after being treated with DES, such as diabetic, small vessel, and bifurcation lesion.

Based on a number of clinical reports, concerns have been raised about an increased risk of stent thrombosis with DES compared to BMS. Stent thrombosis is an uncommon but often devastating complication of coronary stent implantation. Numerous studies have sought to determine the causes of stent thrombosis, as well as any predictors of risk. Premature discontinuation of antiplatelet therapy is strongly associated with the development of stent thrombosis. The delayed healing of the endothelium by the currently available DES due to potent antiproliferative effect of the drugs are other possible causes of late stent thrombosis.

Of the currently approved drug-eluting stents, the TAXAS stent uses the antiproliferative paclitaxel, while the CYPHER sirolimus-eluting coronary stent elutes a substance that limits the overgrowth of normal tissue. Some of the remaining problems with current stents include the toxicity of some of the antiproliferatives, and the rather limited shelf life of these products.

Accordingly, there still remains a need for drug-eluting stents that have reduced toxicity and greater shelf life, while offering equal or greater restenosis prevention or amelioration performance.

### SUMMARY OF THE INVENTION

The present invention relates to a system and device for preventing stenosis and/or restenosis after an invasive procedure in a body vessel or cavity having an inner wall surface, the system comprising inserting a device coated with a growth arresting, lipid-derived, bioactive substance at a desired location along the inner wall surface of the body vessel or cavity. The present invention provides for the use of c-Jun aminoterminal kinase inhibitor of either JNK 1 and/or JNK 2 (“JNK Inhibitor”) and certain analogs as restenosis inhibitors, incorporated into a stent.

Included in the present invention is a stent for implantation into body tissue, preferably comprising a surface and a coating disposed on the surface, wherein the coating comprises at least one JNK Inhibitor.

The JNK Inhibitor may be selected from any such compositions, such as pyrazoloanthrone and derivatives thereof, such as those described in United States Patent Application Nos. 20040176434 and 20040072888 (hereby incorporated by reference), and including by example anthra(1,9-cd)pyrazol-6(2H)-one 1,9-pyrazoloanthrone or analogue thereof (identified as SP600125, commercially available from A.G. Scientific or San Diego, Calif.). Anthra(1,9-cd)pyrazol-6(2H)-one1,9-pyrazoloanthrone (SP600125) (described in SP600125, an anthrapyrazolone inhibitor of Jun N-terminal kinases: B. L. Bennett, et al.; Proc. Natl. Acad. Sci. U.S.A. 98, 13681 (2001); hereby incorporated herein by reference), a pharmacological inhibitor of the c-Jun N-terminal kinase (JNK), can reduce plaque formation in animal model. See Requirement of JNK2 for Scavenger Receptor A-Mediated Foam Cell Formation in Atherogenesis: R. Ricci, et al.; Science 306, 1558 (2004), which is hereby incorporated herein by reference.

SP600125 (chemical formula: C₁₄H₈N₂O) is a potent, cell permeable, selective, and reversible inhibitor of c-Jun N-terminal kinase (JNK) (IC50=40 nM for JNK-1 and JNK-2 and 90 nM for JNK-3), and exhibits over 300-fold greater selectivity for JNK as compared to ERK1 and p38. It is described in B. L. Bennett, et al.; PNAS 98, 13681 (2001), which is hereby incorporated herein by reference.

Using SP600125 as the main drug or in combination with at least one other drug (particularly at a lower dosage of sirolimus) on the drug-eluting stent (DES) of the present invention, will expand the efficacy and safety beyond that in current DES systems.

The stent may be made in accordance with techniques known and used in the art for making drug-eluting stents, especially those adapted to elute relatively hydrophobic materials. Examples are discussed in The Handbook of Drug-Eluting Stents, by Ong, Lemos, Gerschlick and Serruys, Martin Dunitz Ltd. (2005), hereby incorporated herein by reference, and as described in the patents referenced herein.

The stent coating may also be in the form of a polymer containing the JNK inhibitor(s). Acceptable polymers may be biodegradable or non-biodegradable. It is preferred that the polymer forms a biocompatible matrix to allow elution of the anthra(1,9-cd)pyrazol-6(2H)-one 1,9-pyrazoloanthrone or analogue thereof. Other stents that may be used include stents of biodegradable magnesium.

While any concentration of the JNK inhibitor(s) may be used in the polymer with due regard to the release rate and intended vascular environment, in most cases the coating is preferably adapted to release a dosage sufficient to inhibit at least 50% of the enzyme activity (typically measured in vitro), such as at least about 5 nanograms of anthra(1,9-cd)pyrazol-6(2H)-one 1,9-pyrazoloanthrone or analogue thereof per milliliter of blood volume at a selected stent implantation site, and preferably within a range of from about 5 to about 10 nanograms of anthra(1,9-cd)pyrazol-6(2H)-one 1,9-pyrazoloanthrone or analogue thereof per milliliter of blood volume at a selected angioplasty or stent implantation site. The concentration should be sufficient to release a dosage of anthra(1,9-cd)pyrazol-6(2H)-one 1,9-pyrazoloanthrone or analogue thereof sufficient to inhibit the phosphorylation of c-Jun and the expression of at least one of the inflammatory genes COX-2, IL-2, IFN-g and TNF-a (IC50=5-10 mM) in Jurkat T cells, preferably at a level of I.C. 50 (that being sufficient to reduce the activity of the enzyme at least 50 percent).

The present invention also includes a stent as described herein for implantation into body tissue comprising an open-ended tubular structure having a sidewall with apertures therein, wherein the sidewall comprises an outer surface having a coating disposed thereon; the coating comprises anthra (1,9-cd)pyrazol-6(2H)-one 1,9-pyrazoloanthrone or analogue thereof and a polymer, and the coating releases a dosage of about 5 to 10 nanograms of anthra (1,9-cd)pyrazol-6(2H)-one 1,9-pyrazoloanthrone or analogue thereof per milliliter of blood volume at a selected stent implantation site.

The present invention also includes a method of treating or inhibiting restenosis comprising administering to an individual in need thereof an effective amount of an active ingredient selected from the group consisting of at least one c-Jun amino terminal kinase inhibitor, through insertion into the individual of a drug-eluting stent comprising said active ingredient.

It is preferred that the c-Jun inhibitor comprises anthrax (1,9-cd)pyrazol-6(2H)-one 1,9-pyrazoloanthrone or analogue thereof. The dosage may be any effective amount as described above, and typically is administered at a dosage level of that at least that sufficient to reduce the activity of the JNK enzyme.

The JNK inhibitor may be administered contemporaneous with a stent placement, the day of angioplasty procedure or placement, or even after such procedure or placement.

In another aspect, the invention provides a method of treating a mammalian subject to prevent stenosis or restenosis of a blood vessel, comprising the step of administering to a mammalian subject in need of treatment to prevent stenosis or restenosis of a blood vessel a composition comprising a JNK inhibitor, in an amount effective to prevent stenosis or restenosis of the blood vessel, by implanting an intravascular stent in the mammalian subject, where the stent is coated or impregnated with the composition as described herein.

Exemplary materials for constructing a drug-coated or drug-impregnated stent are described in literature cited herein and reviewed in Lincoff et al., Circulation, 90: 2070-2084 (1994), incorporated herein by reference.

In another preferred embodiment, the composition comprises microparticles composed of biodegradable polymers such as PGLA, non-degradable polymers, or biological polymers (e.g., starch) which particles encapsulate or are impregnated by the JNK inhibitor. Such particles are delivered to the intravascular wall using, e.g., an infusion angioplasty catheter. Other techniques for achieving locally sustained drug delivery are reviewed in Wilensky et al., Trends Caridovasc. Med., 3:163-170 (1993), incorporated herein by reference.

Administration via one or more intravenous injections subsequent to the angioplasty, bypass or stent-inserting procedure also is contemplated.

In yet another embodiment, the invention provides the use of a JNK inhibitor for the manufacture of a medicament for the treatment or prevention of stenosis or restenosis of a blood vessel. It is preferred that the medicament include at least one other antiproliferative or anti-inflammatory agent. One of the advantages of this embodiment of the present invention is that these additional agents may be used at concentrations lower than that is stents currently in use. For instance, the stent may use an antiproliferative, such as paclitaxel, at a dosage level lower than that in the TAXAS stent. It may also contain sirolimus, used at a dosage level lower than that currently used in the CYPHER sirolimus-eluting coronary stent.

### DETAILED DESCRIPTION OF THE PREFERRED EMBODIMENT

The present invention is based on the discovery that when one or more JNK inhibitor is incorporated into a stent to be administered through elution to a mammal that has suffered a vascular trauma, such as the trauma that can occur during conventional balloon angioplasty procedures or stent implantation, restenosis of the injured vessel is reduced or eliminated.

The JNK inhibitor may be incorporated into a stent using a polymeric matrix that is used to coat the stent body, in accordance with designs known and used in the art.

As the JNK inhibitor used in accordance with the present invention includes pyrazoloanthrone and derivatives thereof, one may use any polymer or combinations thereof adapted to contain and elute substances of this type. Suitable polymers may include hydrophobic polymers or mixtures of polymers or co-polymers having some hydrophobic character. Examples include a pegylated styrenic block copolymer matrix as described in U.S. Pat. No. 6,918,929, hereby incorporated herein by reference.

The concentration of the pyrazoloanthrone or derivative may be provided in the polymeric matrix so as to provide an effective dosage to tissue in the region of the stent site. The drug-polymer coating may comprise between 0.5 percent and 50 percent of the pyrazoloanthrone or derivative by weight. The drug-polymer coating typically has a thickness between 0.5 microns and 20 microns on the stent surface.

In the preferred embodiment, the stent of the present invention is provided with sufficient JNK inhibitor (i.e., an inhibitor of either JNK1 or JNK2) sufficient to provide and I.C. 50 level; an amount sufficient to inhibit 50% of the JNK enzyme.

It is also preferred that the stent contain at least one additional active ingredient selected from the group consisting of antiproliferatives, most preferably at levels lower than that used in current stent formulations. The stent may also contain additional anti-inflammatory agents. One of the advantages of this embodiment is that lower levels of those active ingredients, such as antiproliferatives, may be used in combination while lowering the overall toxic effect of the stent. The combination of the JNK inhibitor(s) with an antiproliferative will be able to achieve better results that the use of the JNK inhibitor(s) alone, while having overall reduced toxicity associated higher dosage levels of antiproliferatives as in current stent formulations.

The preferred concentration of the JNK inhibitor is that effective to provide a concentration in the range of from about 5 to about 10 nanograms per milliliter at the site of the stent.

The methods and devices described above can be accomplished with many embodiments of stents. Additionally, many stent materials and ancillary drug compounds may be substituted for the supplementary drugs described. Thus, while the preferred embodiments of the devices and methods have been described in reference to the environment in which they were developed, they are merely illustrative of the principles of the inventions. Other embodiments and configurations may be devised without departing from the spirit of the inventions and the scope of the appended claims.

### REFERENCES

The following references are hereby incorporated herein by reference:

PAT. NO.
1 U.S. Pat. No. 6,906,050 Substituted Porphyrin and azaporphyrin derivatives and their use in photodynamic therapy, radioimaginq and MRI diagnosis
2 U.S. Pat. No. 6,827,926 Metallotetrapyrrolic photosensitizing agents for use in photodynamic therapy
3 U.S. Pat. No. 6,824,561 Implantable system with drug-eluting cells for on-demand local drug delivery
4 U.S. Pat. No. 6,716,242 Pulmonary vein stent and method for use
5 U.S. Pat. No. 6,624,138 Drug-loaded biological material chemically treated with genipin
6 U.S. Pat. No. 6,206,914 Implantable system with drug-eluting cells for on-demand local drug delivery
7 U.S. Pat. No. 6,103,705 Pharmaceutical composition comprising a compound having anti-Xa activity and a platelet aggregation antagonist compound
8 U.S. Pat. No. 5,470,307 Catheter system for controllably releasing a therapeutic agent at a remote tissue site

PAT.

NO.
1 U.S. Pat. No. 7,064,211 Hemiasterlin derivatives and uses thereof
2 U.S. Pat. No. 6,852,712 Quinoline and quinoxaline compounds which inhibit platelet-derived growth factor and/or p56lck tyrosine kinases
3 U.S. Pat. No. 6,846,815 Quinoline and quinoxaline compounds which inhibit platelet-derived growth factor and/or p56lck tyrosine kinases
4 U.S. Pat. No. 6,821,962 Quinoline and quinoxaline compounds which inhibit platelet-derived growth factor and/or p56lck tyrosine kinases
5 U.S. Pat. No. 6,696,434 Quinoline and quinoxaline compounds which inhibit platelet-derived growth factor and/or p56lck tyrosine kinases
6 U.S. Pat. No. 6,528,526 Quinoline and quinoxaline compounds which inhibit platelet-derived growth factor and/or p56lck tyrosine kinases
7 U.S. Pat. No. 6,524,347 Quinoline and quinoxaline compounds which inhibit platelet-derived growth factor and/or p56lck tyrosine kinases
8 U.S. Pat. No. 6,482,834 Quinoline and quinoxaline compounds which inhibit platelet-derived growth factor and/or p56lck tyrosine kinases
9 U.S. Pat. No. 6,245,760 Quinoline and quinoxaline compounds which inhibit platelet-derived growth factor and/or p56lck tyrosine kinases
10 U.S. Pat. No. 6,180,632 Quinoline and quinoxaline compounds which inhibit platelet-derived growth factor and/or p56lck tyrosine kinases
11 U.S. Pat. No. 6,159,978 Quinoline and quinoxaline compounds which inhibit platelet-derived growth factor and/or p56.sup.lck tyrosine kinases
12 U.S. Pat. No. 5,470,307 Catheter system for controllably releasing a therapeutic agent at a remote tissue site
1 U.S. Pat. No. 7,074,401 Methods, devices, and compositions for lysis of occlusive blood clots while sparing wound sealing clots
2 U.S. Pat. No. 7,070,616 Implantable valvular prosthesis
3 U.S. Pat. No. 7,064,211 Hemiasterlin derivatives and uses thereof
4 U.S. Pat. No. 7,063,884 Stent coating
5 U.S. Pat. No. 7,063,720 Covered stent with controlled therapeutic agent diffusion
6 U.S. Pat. No. 7,056,591 Hydrophobic biologically absorbable coatings for drug delivery devices and methods for fabricating the same
7 U.S. Pat. No. 7,056,339 Drug delivery platform
8 U.S. Pat. No. 7,056,338 Therapeutic agent delivery device with controlled therapeutic agent release rates
9 U.S. Pat. No. 7,055,237 Method of forming a drug eluting stent
10 U.S. Pat. No. 7,052,516 Spinal disc annulus reconstruction method and deformable spinal disc annulus stent
11 U.S. Pat. No. 7,052,513 Three-dimensional braided covered stent
12 U.S. Pat. No. 7,048,962 Stent coating device
13 U.S. Pat. No. 7,048,714 Drug eluting medical device with an expandable portion for drug release
14 U.S. Pat. No. 7,041,127 Textured and drug eluting coronary artery stent
15 U.S. Pat. No. 7,040,485 Method and apparatus for packaging a drug-device combination product
16 U.S. Pat. No. 7,037,332 Medical device with coating that promotes endothelial cell adherence
17 U.S. Pat. No. 7,029,493 Stent with enhanced crossability
18 U.S. Pat. No. 7,026,356 Fatty acid analogues for the treatment of diseases caused by the pathological proliferation of smooth muscle cells
19 U.S. Pat. No. 7,026,355 Use of rhein or diacerhein compounds for the treatment or prevention of vascular diseases
20 U.S. Pat. No. 7,022,372 Compositions for coating implantable medical devices
21 U.S. Pat. No. 7,011,643 Grafted network incorporating a multiple channel fluid flow connector
22 D516,723 Stent wall structure
23 U.S. Pat. No. 7,008,667 Bioactive agent release coating
24 U.S. Pat. No. 7,008,411 Method and apparatus for treating vulnerable plaque
25 U.S. Pat. No. 7,008,397 Cardiac implant and methods
26 U.S. Pat. No. 7,005,137 Coating for implantable medical devices
27 U.S. Pat. No. 7,004,970 Methods and devices for spinal disc annulus reconstruction and repair
28 U.S. Pat. No. 7,001,421 Stent with phenoxy primer coating
29 U.S. Pat. No. 6,996,952 Method for improving stability and effectivity of a drug-device combination product
30 U.S. Pat. No. 6,991,617 Vascular treatment method and device
31 U.S. Pat. No. 6,991,615 Grafted network incorporating a multiple channel fluid flow connector
32 U.S. Pat. No. 6,986,751 Grafted network incorporating a multiple channel fluid flow connector
33 U.S. Pat. No. 6,971,998 Implant delivery catheter system and methods for its use
34 U.S. Pat. No. 6,971,813 Contact coating of prostheses
35 U.S. Pat. No. 6,970,742 Method for detecting, diagnosing, and treating cardiovascular disease
36 U.S. Pat. No. 6,951,053 Method of manufacturing a prosthesis
37 U.S. Pat. No. 6,939,863 Prevention of atherosclerosis and restenosis
38 U.S. Pat. No. 6,939,376 Drug-delivery endovascular stent and method for treating restenosis
39 U.S. Pat. No. 6,939,345 Method for reducing restenosis in the presence of an intravascular stent
40 U.S. Pat. No. 6,936,065 Stent delivery system having a fixed guidewire
41 U.S. Pat. No. 6,932,091 Method for surgically restoring coronary blood vessels
42 U.S. Pat. No. 6,926,919 Method for fabricating a coating for a medical device
43 U.S. Pat. No. 6,918,929 Drug-polymer coated stent with pegylated styrenic block copolymers
44 U.S. Pat. No. 6,906,050 Substituted porphyrin and azaporphyrin derivatives and their use in photodynamic therapy, radioimaging and MRI diagnosis
45 U.S. Pat. No. 6,904,658 Process for forming a porous drug delivery layer
46 U.S. Pat. No. 6,890,546 Medical devices containing rapamycin analogs
47 U.S. Pat. No. 6,860,851 Vulnerable plague diagnosis and treatment
48 U.S. Pat. No. 6,852,712 Quinoline and quinoxaline compounds which inhibit platelet-derived growth factor and/or p56lck tyrosine kinases
49 U.S. Pat. No. 6,852,123 Micro structure stent configurations
50 U.S. Pat. No. 6,846,815 Quinoline and quinoxaline compounds which inhibit platelet-derived growth factor and/or p56lck tyrosine kinases
51 U.S. Pat. No. 6,830,747 Biodegradable copolymers linked to segment with a plurality of functional groups
52 U.S. Pat. No. 6,827,926 Metallotetrapyrrolic photosensitizing agents for use in photodynamic therapy
53 U.S. Pat. No. 6,824,561 Implantable system with drug-eluting cells for on-demand local drug delivery
54 U.S. Pat. No. 6,824,559 Ethylene-carboxyl copolymers as drug delivery matrices
55 U.S. Pat. No. 6,821,962 Quinoline and quinoxaline compounds which inhibit platelet-derived growth factor and/or p56lck tyrosine kinases
56 U.S. Pat. No. 6,818,016 Methods for coating stents with DNA and expression of recombinant genes from DNA coated stents in vivo
57 U.S. Pat. No. 6,805,898 Surface features of an implantable medical device
58 U.S. Pat. No. 6,805,876 Phosphate based biodegradable Polymers
59 U.S. Pat. No. 6,797,727 Use of rhein or diacerhein compounds for the treatment or prevention of vascular diseases
60 U.S. Pat. No. 6,783,793 Selective coating of medical devices
61 U.S. Pat. No. 6,764,507 Expandable medical device with improved spatial distribution
62 U.S. Pat. No. 6,764,505 Variable surface area stent
63 U.S. Pat. No. 6,761,734 Segmented balloon catheter for stenting bifurcation lesions
64 U.S. Pat. No. 6,746,481 Implatable device including a polyamino acid component
65 U.S. Pat. No. 6,726,923 Apparatus and methods for preventing or treating failure of hemodialysis vascular access and other vascular grafts
66 U.S. Pat. No. 6,725,901 Methods of manufacture of fully consolidated or porous medical devices
67 U.S. Pat. No. 6,716,444 Barriers for polymer-coated implantable medical devices and methods for making the same
68 U.S. Pat. No. 6,716,242 Pulmonary vein stent and method for use
69 U.S. Pat. No. 6,712,767 Ultrasonic imaging devices and methods of fabrication
70 U.S. Pat. No. 6,702,850 Multi-coated drug-eluting stent for antithrombosis and antirestenosis
71 U.S. Pat. No. 6,696,434 Quinoline and quinoxaline compounds which inhibit platelet-derived growth factor and/or p56lck tyrosine kinases
72 U.S. Pat. No. 6,689,803 Compositions and methods for treating surgical adhesions
73 U.S. Pat. No. 6,656,216 Composite stent with regioselective material
74 U.S. Pat. No. 6,648,881 Method for reducing arterial restenosis in the presence of an intravascular stent
75 U.S. Pat. No. 6,635,070 Apparatus and methods for capturing particulate material within blood vessels
76 U.S. Pat. No. 6,635,027 Method and apparatus for intramural delivery of a substance
77 U.S. Pat. No. 6,626,940 Medical device activation system
78 U.S. Pat. No. 6,626,939 Stent-graft with bioabsorbable structural support
79 U.S. Pat. No. 6,624,138 Drug-loaded biological material chemically treated with genipin
80 U.S. Pat. No. 6,623,521 Expandable stent with sliding and locking radial elements
81 U.S. Pat. No. 6,620,194 Drug coating with topcoat
82 U.S. Pat. No. 6,592,617 Three-dimensional braided covered stent
83 U.S. Pat. No. 6,537,247 Shrouded strain relief medical balloon device and method of use
84 U.S. Pat. No. 6,537,195 Combination x-ray radiation and drug delivery devices and methods for inhibiting hyperplasia
85 U.S. Pat. No. 6,528,526 Quinoline and quinoxaline compounds which inhibit platelet-derived growth factor and/or p56lck tyrosine kinases
86 U.S. Pat. No. 6,524,347 Quinoline and quinoxaline compounds which inhibit platelet-derived growth factor and/or p56lck tyrosine kinases
87 U.S. Pat. No. 6,519,488 Method and system for reducing arterial restenosis in the presence of an intravascular stent
88 U.S. Pat. No. 6,515,016 Composition and methods of paclitaxel for treating psoriasis
89 U.S. Pat. No. 6,511,507 Article with biocompatible coating
90 U.S. Pat. No. 6,500,859 Method for treating atherosclerosis or restenosis using microtubule stabilizing agent
91 U.S. Pat. No. 6,495,579 Method for treating multiple sclerosis
92 RE37,933 Viral vectors and their use for treating hyperproliferative disorders, in particular restenosis
93 U.S. Pat. No. 6,482,834 Quinoline and quinoxaline compounds which inhibit platelet-derived growth factor and/or p56lck tyrosine kinases
94 U.S. Pat. No. 6,478,776 Implant delivery catheter system and methods for its use
95 U.S. Pat. No. 6,429,232 Method of treating atherosclerosis or restenosis using microtubule stabilizing agent
96 U.S. Pat. No. 6,428,569 Micro structure stent configurations
97 U.S. Pat. No. 6,417,232 Fatty acid analogues for the treatment of Primary and secondary restenosis
98 U.S. Pat. No. 6,403,635 Method of treating atherosclerosis or restenosis using microtubule stabilizing agent
99 U.S. Pat. No. 6,395,023 Prosthesis with biodegradable surface coating and method for making same
100 U.S. Pat. No. 6,378,218 Methods and apparatus for making a drug infusion device
101 U.S. Pat. No. 6,342,068 Three-dimensional braided stent
102 U.S. Pat. No. 6,340,367 Radiopaque markers and methods of using the same
103 U.S. Pat. No. 6,317,615 Method and system for reducing arterial restenosis in the presence of an intravascular stent
104 U.S. Pat. No. 6,284,305 Drug coating with topcoat
105 U.S. Pat. No. 6,251,135 Radiopaque marker system and method of use
106 U.S. Pat. No. 6,245,760 Quinoline and quinoxaline compounds which inhibit platelet-derived growth factor and/or p56lck tyrosine kinases
107 U.S. Pat. No. 6,245,103 Bioabsorbable self-expanding stent
108 U.S. Pat. No. 6,218,016 Lubricious, drug-accommodating coating
109 U.S. Pat. No. 6,206,914 Implantable system with drug-eluting cells for on-demand local drug delivery
110 U.S. Pat. No. 6,200,302 Hypodermic needle for percutaneous drug delivery
111 U.S. Pat. No. 6,180,632 Quinoline and Quinoxaline compounds which inhibit platelet-derived growth factor and/or p56lck tyrosine kinases
112 U.S. Pat. No. 6,174,330 Bioabsorbable marker having radiopaque constituents
113 U.S. Pat. No. 6,159,978 Quinoline and quinoxaline compounds which inhibit platelet-derived growth factor and/or p56.sup.lck tyrosine kinases
114 U.S. Pat. No. 6,159,488 Intracoronary stents containing quinazolinone derivatives
115 U.S. Pat. No. 6,120,536 Medical devices with long term non-thrombogenic coatings
116 U.S. Pat. No. 6,103,705 Pharmaceutical composition comprising a compound having anti-Xa activity and a platelet aggregation antagonist compound
117 U.S. Pat. No. 6,099,562 Drug coating with topcoat
118 U.S. Pat. No. 6,080,190 intraluminal stent
119 U.S. Pat. No. 6,004,346 Intralumenal drug eluting prosthesis
120 U.S. Pat. No. 5,997,468 Intraluminal drug eluting prosthesis method
121 U.S. Pat. No. 5,980,564 Bioabsorbable implantable endoprosthesis with reservoir
122 U.S. Pat. No. 5,980,551 Composition and method for making a biodegradable drug delivery stent
123 U.S. Pat. No. 5,968,091 Stents and stent grafts having enhanced hoop strength and methods of making the same
124 U.S. Pat. No. 5,957,971 Intraluminal stent
125 U.S. Pat. No. 5,951,586 Intraluminal stent
126 U.S. Pat. No. 5,900,433 Vascular treatment method and apparatus
127 U.S. Pat. No. 5,893,840 Releasable microcapsules on balloon catheters
128 U.S. Pat. No. 5,893,839 Timed-release localized drug delivery by percutaneous administration
129 U.S. Pat. No. 5,871,535 Intralumenal drug eluting prosthesis
130 U.S. Pat. No. 5,851,521 Viral vectors and their use for treating hyperproliferative disorders, in particular restenosis
131 U.S. Pat. No. 5,851,231 Intralumenal drug eluting prosthesis
132 U.S. Pat. No. 5,851,217 Intralumenal drug eluting prosthesis
133 U.S. Pat. No. 5,849,034 Intraluminal stent
134 U.S. Pat. No. 5,837,008 Intravascular stent and method
135 U.S. Pat. No. 5,824,048 Method for delivering a therapeutic substance to a body lumen
136 U.S. Pat. No. 5,800,507 Intraluminal stent
137 U.S. Pat. No. 5,799,384 Intravascular radially expandable stent
138 U.S. Pat. No. 5,779,732 Method and apparatus for implanting a film with an expandable stent
139 U.S. Pat. No. 5,776,184 Intravascular stent and method
140 U.S. Pat. No. 5,725,567 Method of making a intralumenal drug eluting prosthesis
141 U.S. Pat. No. 5,718,159 Process for manufacturing three-dimensional braided covered stent
142 U.S. Pat. No. 5,697,967 Drug eluting stent
143 U.S. Pat. No. 5,681,278 Coronary vasculature treatment method
144 U.S. Pat. No. 5,679,400 Intravascular stent and method
145 U.S. Pat. No. 5,651,174 Intravascular radially expandable stent
146 U.S. Pat. No. 5,634,895 Apparatus and method for transpericardial delivery of fluid
147 U.S. Pat. No. 5,628,785 Bioelastomeric stent
148 U.S. Pat. No. 5,624,411 Intravascular stent and method
149 U.S. Pat. No. 5,616,608 Method of treating atherosclerosis or restenosis using microtubule stabilizing agent
150 U.S. Pat. No. 5,613,981 Bidirectional dual sinusoidal helix stent
151 U.S. Pat. No. 5,599,352 Method of making a drug eluting stent
152 U.S. Pat. No. 5,591,227 Drug eluting stent
153 U.S. Pat. No. 5,591,224 Bioelastomeric stent
154 U.S. Pat. No. 5,571,166 Method of making an intraluminal stent
155 U.S. Pat. No. 5,554,182 Method for preventing restenosis
156 U.S. Pat. No. 5,545,208 Intralumenal drug eluting prosthesis
157 U.S. Pat. No. 5,510,077 Method of making an intraluminal stent
158 U.S. Pat. No. 5,470,307 Catheter system for controllably releasing a therapeutic agent at a remote tissue site
159 U.S. Pat. No. 5,464,650 Intravascular stent and method
160 U.S. Pat. No. 5,443,496 Intravascular radially expandable stent
161 U.S. Pat. No. 5,370,614 Method for making a drug delivery balloon catheter
162 U.S. Pat. No. 5,324,261 Drug delivery balloon catheter with line of weakness
163 U.S. Pat. No. 5,282,823 Intravascular radially expandable stent
164 U.S. Pat. No. 5,102,402 Releasable coatings on balloon catheters
1 U.S. Pat. No. 7,070,616 Implantable valvular prosthesis
2 U.S. Pat. No. 7,063,884 Stent coating
3 U.S. Pat. No. 7,063,720 Covered stent with controlled therapeutic agent diffusion
4 U.S. Pat. No. 7,056,339 Drug delivery platform
5 U.S. Pat. No. 7,055,237 Method of forming a drug eluting stent
6 U.S. Pat. No. 7,052,516 Spinal disc annulus reconstruction method and deformable spinal disc annulus stent
7 U.S. Pat. No. 7,048,962 Stent coating device
8 U.S. Pat. No. 7,041,127 Textured and drug eluting coronary artery stent
9 U.S. Pat. No. 7,037,332 Medical device with coating that promotes endothelial cell adherence
10 U.S. Pat. No. 7,029,493 Stent with enhanced crossability
11 U.S. Pat. No. 7,005,137 Coating for implantable medical devices
12 U.S. Pat. No. 7,004,970 Methods and devices for spinal disc annulus reconstruction and repair
13 U.S. Pat. No. 7,001,421 Stent with phenoxy primer coating
14 U.S. Pat. No. 6,991,617 Vascular treatment method and device
15 U.S. Pat. No. 6,939,376 Drug-delivery endovascular stent and method for treating restenosis
16 U.S. Pat. No. 6,939,345 Method for reducing restenosis in the presence of an intravascular stent
17 U.S. Pat. No. 6,936,065 Stent delivery system having a fixed guidewire
18 U.S. Pat. No. 6,932,091 Method for surgically restoring coronary blood vessels
19 U.S. Pat. No. 6,918,929 Drug-polymer coated stent with pegylated styrenic block copolymers
20 U.S. Pat. No. 6,904,658 Process for forming a porous drug delivery layer
21 U.S. Pat. No. 6,824,559 Ethylene-carboxyl copolymers as drug delivery matrices
22 U.S. Pat. No. 6,805,898 Surface features of an implantable medical device
23 U.S. Pat. No. 6,764,505 Variable surface area stent
24 U.S. Pat. No. 6,716,242 Pulmonary vein stent and method for use
25 U.S. Pat. No. 6,702,850 Multi-coated drug-eluting stent for antithrombosis and antirestenosis
26 U.S. Pat. No. 6,656,216 Composite stent with regioselective material
27 U.S. Pat. No. 6,648,881 Method for reducing arterial restenosis in the presence of an intravascular stent
28 U.S. Pat. No. 6,626,939 Stent-graft with bioabsorbable structural support
29 U.S. Pat. No. 6,623,521 Expandable stent with sliding and locking radial elements
30 U.S. Pat. No. 6,537,195 Combination x-ray radiation and drug delivery devices and methods for inhibiting hyperplasia
31 U.S. Pat. No. 6,519,488 Method and system for reducing arterial restenosis in the presence of an intravascular stent
32 U.S. Pat. No. 6,317,615 Method and system for reducing arterial restenosis in the Presence of an intravascular stent
33 U.S. Pat. No. 6,245,103 Bioabsorbable self-expanding stent
34 U.S. Pat. No. 6,159,488 Intracoronary stents containing quinazolinone derivatives
35 U.S. Pat. No. 6,120,536 Medical devices with long term non-thrombogenic coatings
36 U.S. Pat. No. 6,080,190 Intraluminal stent
37 U.S. Pat. No. 5,980,551 Composition and method for making a biodegradable drug delivery stent
38 U.S. Pat. No. 5,968,091 Stents and stent grafts having enhanced hoop strength and methods of making the same
39 U.S. Pat. No. 5,957,971 Intraluminal stent
40 U.S. Pat. No. 5,951,586 Intraluminal stent
41 U.S. Pat. No. 5,893,840 Releasable microcapsules on balloon catheters
42 U.S. Pat. No. 5,849,034 Intraluminal stent
43 U.S. Pat. No. 5,837,008 Intravascular stent and method
44 U.S. Pat. No. 5,824,048 Method for delivering a therapeutic substance to a body lumen
45 U.S. Pat. No. 5,800,507 Intraluminal stent
46 U.S. Pat. No. 5,799,384 Intravascular radially expandable stent
47 U.S. Pat. No. 5,779,732 Method and apparatus for implanting a film with an expandable stent
48 U.S. Pat. No. 5,776,184 Intravascular stent and method
49 U.S. Pat. No. 5,697,967 Drug eluting stent
50 U.S. Pat. No. 5,679,400 Intravascular stent and method
51 U.S. Pat. No. 5,651,174 Intravascular radially expandable stent
52 U.S. Pat. No. 5,628,785 Bioelastomeric stent
53 U.S. Pat. No. 5,624,411 Intravascular stent and method
54 U.S. Pat. No. 5,613,981 Bidirectional dual sinusoidal helix stent
55 U.S. Pat. No. 5,599,352 Method of making a drug eluting stent
56 U.S. Pat. No. 5,591,227 Drug eluting stent
57 U.S. Pat. No. 5,591,224 Bioelastomeric stent
58 U.S. Pat. No. 5,571,166 Method of making an intraluminal stent
59 U.S. Pat. No. 5,554,182 Method for preventing restenosis
60 U.S. Pat. No. 5,510,077 Method of making an intraluminal stent
61 U.S. Pat. No. 5,464,650 Intravascular stent and method
62 U.S. Pat. No. 5,443,496 Intravascular radially expandable stent
63 U.S. Pat. No. 5,282,823 Intravascular radially expandable stent

PAT. NO. Title
1 U.S. Pat. No. 7,067,111 T Ethylenedicysteine (EC)-drug conjugates, compositions and methods for tissue specific disease imaging
2 U.S. Pat. No. 6,998,475 T Variants of traf2 which act as an inhibitor of tnf-alpha (tnf.alpha.) signaling Pathway
3 U.S. Pat. No. 6,962,904 T Elastin peptide analogs and uses thereof
4 U.S. Pat. No. 6,682,545 T System and device for preventing restenosis in body vessels
1 20040176434 Methods for treating inflammatory conditions or inhibiting JNK
2 20040072888 Methods for treating inflammatory conditions or inhibiting JNK

What is claimed is:

1. A stent for implantation into body tissue comprising a surface and a coating disposed on the surface, wherein the coating comprises at least one c-Jun aminoterminal kinase inhibitor.

2. The stent according to claim 1 wherein said c-Jun inhibitor comprises anthra(1,9-cd)pyrazol-6(2H)-one 1,9-pyrazoloanthrone or analogue thereof.

3. The stent according to claim 1 wherein said coating comprises a polymer containing said c-Jun aminoterminal kinase inhibitor.

4. The stent according to claim 2 wherein the polymer is non-biodegradable.

5. The stent according to claim 2 wherein the polymer is biodegradable.

6. The stent according to claim 2 wherein said coating comprises a polymer containing said anthra(1,9-cd)pyrazol-6(2H)-one 1,9-pyrazoloanthrone or analogue thereof.

7. The stent according to claim 6 wherein the polymer is non-biodegradable.

8. The stent according to claim 6 wherein the polymer is biodegradable.

6. The stent according to claim 2 wherein the coating is adapted to release a dosage of at least about 5 nanograms of anthra(1,9-cd)pyrazol-6(2H)-one 1,9-pyrazoloanthrone or analogue thereof per milliliter of blood volume at a selected stent implantation site.

7. The stent according to claim 2 wherein the coating is adapted to release a dosage of about 5 to about 10 nanograms of anthra(1,9-cd)pyrazol-6(2H)-one 1,9-pyrazoloanthrone or analogue thereof per milliliter of blood volume at a selected stent implantation site.

8. The stent according to claim 2 wherein the coating is adapted to release a dosage of anthra(1,9-cd)pyrazol-6(2H)-one 1,9-pyrazoloanthrone or analogue thereof sufficient to inhibit the phosphorylation of c-Jun and the expression of at least one of the inflammatory genes COX-2, IL-2, IFN-g and TNF-a in Jurkat T cells to a level lower than 50 percent activity.

9. The stent according to claim 1, additionally comprising at least one additional active ingredient selected from the group consisting of anti-inflammatory agents and antiproliferative agents.

10. A stent for implantation into body tissue comprising an open-ended tubular structure having a sidewall with apertures therein, wherein:
(a) the sidewall comprises an outer surface having a coating disposed thereon;
(b) the coating comprises anthra(1,9-cd)pyrazol-6(2H)-one 1,9-pyrazoloanthrone or analogue thereof and a polymer, and (c) the coating releases a dosage of anthra(1,9-cd)pyrazol-6(2H)-one 1,9-pyrazoloanthrone or analogue thereof at a selected stent implantation site sufficient to reduce the activity of a JNK enzyme by at least 50 percent.

11. A method of treating or inhibiting restenosis comprising administering to an individual in need thereof an effective amount of an active ingredient selected from the group consisting of at least one c-Jun aminoterminal kinase inhibitor through insertion into said individual of a drug-eluting stent comprising said active ingredient.

12. The method according to claim 11, wherein said c-Jun inhibitor comprises anthra(1,9-cd)pyrazol-6(2H)-one 1,9-pyrazoloanthrone or analogue thereof.

13. The method according to claim 11, wherein said active ingredient is administered at a dosage level of at least about 5 nanograms of anthra(1,9-cd)pyrazol-6(2H)-one 1,9-pyrazoloanthrone or analogue thereof per milliliter of blood volume at a selected stent implantation site.

14. The method according to claim 11, wherein said active ingredient is administered at a dosage level of in the range of from about 5 to about 10 nanograms of anthra(1,9-cd)pyrazol-6(2H)-one 1,9-pyrazoloanthrone or analogue thereof per milliliter of blood volume at a selected stent implantation site.

15. The method according to claim 11, wherein said active ingredient is administered before an angioplasty procedure.

16. The method according to claim 11, wherein said active ingredient is administered the day of an angioplasty procedure.

17. The method according to claim 11, wherein said active ingredient is administered after an angioplasty procedure.

18. The method according to claim 11, wherein said drug-eluting stent additionally comprises at least one additional active ingredient selected from the group consisting of anti-inflammatory agents and antiproliferative agents.
